(19) United States
(12) Patent Application Publication
(10) Pub. No.: US 2024/0269420 A1
(43) Pub. Date: Aug. 15, 2024

(54) A BREATHING ASSEMBLY

(71) Applicant: FLEXICARE (GROUP) LIMITED, Mountain Ash Mid Glamorgan (GB)

(72) Inventors: Tudor Thomas, Mountain Ash Mid Glamorgan (GB); Daniel Rees-Whippey, Mountain Ash Mid Glamorgan (GB); Ghassem Poormand, Mountain Ash Mid Glamorgan (GB)

(21) Appl. No.: 18/566,858

(22) PCT Filed: Jun. 1, 2022
(86) PCT No.: PCT/GB2022/051398

§ 371 Date: Dec. 4, 2023

(30) Foreign Application Priority Data
Jun. 4, 2021 (GB) 2108038.7
Jan. 14, 2022 (GB) 2200453.5

(51) Int. Cl. A61M 16/08; A61M 16/10

(52) U.S. Cl. CPC A61M 16/0875; A61M 16/0816; A61M 16/1095; A61M 2205/02; A61M 2205/3368

(57) ABSTRACT

A breathing assembly supplying gases to a patient includes a patient end connector, a chamber end connector, and a coaxial conduit assembly extending between the patient end connector and the ventilator end connector. The coaxial conduit assembly includes inner and outer conduits, the inner conduit arranged within the outer conduit so as to define a first flow passage along the coaxial conduit assembly within the inner conduit and a second flow passage along the coaxial conduit assembly between the inner conduit and outer conduit. Inspiratory gases are conveyed along the first flow passage and expiratory gases are conveyed along the second flow passage, in use, and the outer conduit is at least partially formed from a water permeable material configured to allow water to flow therethrough.

### FIELD

The present teachings relate to a breathing assembly.

### BACKGROUND

Patients with compromised ability to breathe autonomously can have their respiration supported through a breathing system, i.e. through mechanical ventilation. Mechanical ventilation utilises externally generated positive air pressure to induce a pressure gradient to the lungs, leading to a flow in of air. This pressure gradient is varied cyclically, in order to mimic the normal respiration of a patient. With mechanical ventilation, the patient must be connected to the source of the pressure change (e.g. a ventilator) through a breathing system.

In such assisted/support breathing, particularly in medical applications, the breathing system needs to be capable of supplying gases, known as inspiratory gases, to a patient at suitable temperature and humidity levels. In known breathing systems, the high humidity of the conveyed gases can result in the build-up of condensation within the conduits.

The present teachings seek to overcome or at least mitigate one or more problems associated with the prior art.

### SUMMARY

According to a first aspect, there is provided a breathing assembly for supplying gases to a patient, the breathing assembly comprising: a patient end connector configured to be connectable to an airway device; a chamber end connector configured to be connectable to a humidification chamber and comprising an inspiratory inlet for receiving humidified inspiratory gases from the humidification chamber; and a coaxial conduit assembly extending between the patient end connector and the ventilator end connector and comprising inner and outer conduits defined by inner and outer conduit wall, the inner conduit wall arranged within the outer conduit wall so as to define a first flow passage along the coaxial conduit assembly within the inner conduit wall and a second flow passage along the coaxial conduit assembly between the inner conduit wall and outer conduit wall, wherein the breathing assembly is configured such that inspiratory gases are conveyed along the first flow passage and expiratory gases are conveyed along the second flow passage, in use, and wherein the outer conduit wall is at least partially formed from a water permeable material configured to allow water to flow therethrough.

It will be appreciated that the airway device may be an infraglottic airway (e.g. endotracheal tube, endobronchial tube, tracheostomy tube), or a supraglottic airway device (e.g. oropharyngeal airway, nasopharyngeal airway, and laryngeal mask airway).

In this arrangement, the second flow passage provides a thermal barrier between the inspiratory gas in the first flow passage and the ambient air, which facilitates maintaining the inspiratory gas at the required temperature.

Providing an outer conduit that is formed from a water permeable material that allows water to flow therethrough has been found to reduce the build-up of water in the second flow passage, and can also reduce the build-up of water in the ventilator.

The water permeable material may be configured to allow flow of liquid water therethrough and configured to restrict flow of respiratory gases therethrough.

The water permeable material may be configured to restrict the flow of liquid water therethrough.

Providing an outer conduit that is formed from a water permeable material that allows water to flow therethrough and that allows, but restricts, the passage of liquid water and/or gases therethrough has been found to reduce the build-up of water and water vapour in the second flow passage.

Substantially all, e.g. an entirety, of the outer conduit wall may be formed from the water permeable material.

This has been found to enhance the breathability of the outer conduit, thereby further reducing the build-up of water and water vapour in the second flow passage.

The outer conduit wall may be self-supporting.

This removes the need for additional structural members to be mounted to the outer conduit to provide the necessary structural stiffness/rigidity.

The outer conduit wall may comprise a reinforcing arrangement.

This provides the outer conduit with improved structural stiffness/rigidity.

The reinforcing arrangement may comprise a corrugated region of the outer conduit wall, optionally wherein the corrugated region extends over an entirety of the elongate length of the outer conduit wall.

This removes the need for additional structural members to be mounted to the outer conduit to provide the necessary structural stiffness/rigidity.

The outer conduit may be configured to support the inner conduit.

The coaxial conduit assembly may comprise a spacing arrangement between the inner conduit wall and the outer conduit wall.

The spacing arrangement may comprise at least one spacing assembly provided along the elongate length of the coaxial conduit assembly.

Each spacing assembly may comprise a plurality of spacing members positioned between the inner conduit wall and the outer conduit wall.

The plurality of spacing members may be equally spaced around the inner conduit wall.

The spacing members may project inwardly (i.e. radially inwardly) from the outer conduit wall.

The outer conduit may define an outer conduit wall having a thickness in the range 0.2 mm to 1.0 mm, optionally in the range 0.3 mm to 0.9 mm.

Providing an outer conduit wall in this range has been found to provide sufficient stiffness/rigidity of the outer conduit whilst providing a suitably water permeable outer conduit wall.

The water permeable material may be formed from an amphiphilic material.

This has been found to enhance the permeability of the outer conduit to water vapour and liquid water, thereby reducing the build-up of water and water vapour in the second flow passage.

The water permeable material may be formed from a hydrophobic and hydrophilic block co-polymer.

The water permeable material may be formed from a hydrophobic and hydrophilic poly (ethylene oxide), poly(butylene terephthalate) (PBT) based block co-polymer.

This has been found to enhance the permeability of the outer conduit to water vapour and liquid water, thereby reducing the build-up of water and water vapour in the second flow passage.

The outer conduit wall may be configured to absorb water vapour and liquid water.

This has been found to further enhance the permeability of the outer conduit to water vapour and liquid water, thereby further reducing the build-up of water and water vapour in the second flow passage.

The inner conduit wall may be insufficiently strong to be self-supporting.

The inner conduit wall may be formed from a thermoplastic elastomer.

The inner conduit wall may be configured to prevent water and water vapour from flowing therethrough.

The inner conduit wall may be configured to prevent gases from flowing therethrough.

This arrangement enables the humidity of the inspiratory gases to be actively, rather than passively, controlled by the breathing assembly.

The inner conduit may comprise a reinforcing arrangement.

Incorporating a reinforcing arrangement enables the thickness of the wall of the inner conduit to be reduced, thus reducing the amount of material used in the inner conduit.

The reinforcing arrangement may comprise a reinforcing rib extending around, e.g. helically around, the inner conduit wall.

This has been found to further improve reinforcement of the inner conduit.

The breathing assembly may comprise a heating arrangement configured and arranged to heat inspiratory gases flowing along the first flow passage, wherein the heating arrangement comprises a first heating member embedded within an inner conduit wall of the inner conduit.

This has been found to improve the packing of the breathing assembly. By arranging the first heating element within the wall of the inner conduit wall, the obstruction to flow of the inspiratory gases is reduced.

The heating arrangement may be configured such that the inspiratory gases maintain an absolute humidity of at least 33 mg/l.

The first heating member may extend around, e.g. spiral around, the inner conduit wall of the inner conduit.

Arranging the first heating element as a spiral around the wall of the inner conduit has been found to provide more uniform heating of the inspiratory gases flowing within the inner conduit.

The first heating member may be configured and arranged to heat inspiratory gases flowing along the first flow passage and to heat expiratory gases flowing along the second flow passage.

Arranging the first heating element so as to heat both inspiratory and expiratory gases can reduce or remove the need for a separate heating arrangement for the expiratory gases.

The first heating member may comprise a heating power in the range 25 to 40 Watts, optionally in the range 27 to 35 Watts, optionally in the range 29 to 33 Watts, for example approximately 31 Watts.

The first heating member may be configured to heat the inspiratory gases to a temperature in the range 36° C. to 41° C., for example in the range 37° C. to 40° C.

The inner conduit wall may comprise a reinforcing rib extending therearound, and wherein the first heating member is embedded within the reinforcing rib.

Arranging the first heating element within the reinforcing rib has been found to improve the packing of the breathing assembly.

The breathing assembly may comprise a catheter mount connected to the patient end connector.

The breathing assembly may comprise a second temperature sensor at or near the patient end connector.

The second temperature sensor may be integrally formed with the patient end connector. Alternately, the second temperature sensor may be separate from, and connectable to, the patient end connector (i.e. the second temperature sensor may be reusable).

The inner conduit wall may comprise a reinforcing rib extending therearound, and wherein one or more electrical wires connected to the second temperature sensor are embedded within the reinforcing rib.

Arranging the electrical wires within the reinforcing rib has been found to improve the packing of the breathing assembly.

The breathing assembly may comprise a first temperature sensor at or near the ventilator end connector.

The first temperature sensor may be integrally formed with the ventilator end connector. Alternately, the first temperature sensor may be separate from, and connectable to, the ventilator end connector (i.e. the first temperature sensor may be reusable).

The heating arrangement may comprise a second heating member configured to heat gases flowing along the second flow passage, wherein the second heating element is either positioned in the second flow passage or is embedded within a wall of the outer conduit.

The second heating member may be configured to heat the outer conduit wall to a predetermined temperature range or a predetermined temperature.

This enables the second heating member to heat the outer conduit wall to a temperature or temperature range so as to maximise its permeability to water.

The second heating member may comprise a heating power in the range 5 to 40 Watts.

The breathing assembly may comprise an expiratory port for conveying expiratory gasses out of the breathing assembly, wherein the expiratory port is provided at or proximate to the patient end connector

The breathing assembly may comprise a flow valve at or near the interface between the coaxial conduit assembly and the patient end connector, the flow valve configured to allow flow of inspiratory gases from the first flow to passage into the patient end connector and to direct expiratory gases flowing from the patient end connector to the coaxial conduit assembly into the second flow passage.

According to a second aspect, there is provided a breathing assembly for supplying gases to a patient, the breathing assembly comprising: a patient end connector configured to be connectable to an air way device; a ventilator end connector configured to be connectable to a humidification chamber and comprising an inspiratory inlet for receiving humidified inspiratory gases from the humidification chamber; a coaxial conduit assembly extending between the patient end connector and the ventilator end connector and comprising inner and outer conduits defined by inner and outer conduit walls, the inner conduit wall arranged within the outer conduit wall so as to define a first flow passage along the coaxial conduit assembly within the inner conduit wall and a second flow passage along the coaxial conduit assembly between the inner conduit wall and outer conduit wall, wherein the first flow passage and second flow passage each convey one of inspiratory gases or expiratory gases, in use; and a heating arrangement for heating gases flowing along the coaxial conduit assembly, wherein the heating arrangement comprises a first heating member embedded within the inner conduit wall, and wherein the first heating member is configured and arranged to heat gases flowing along the first flow passage and the second flow passage.

This has been found to improve the packing of the breathing assembly. By arranging the first heating element within the wall (i.e. on the outer surface of the wall) of the inner conduit, the obstruction to flow of the inspiratory gases is reduced.

Arranging the first heating element so as to heat both inspiratory and expiratory gases can reduce the heat that needs to be supplied by a dedicated expiratory gas heating arrangement.

It will be appreciated that the airway device may be an infraglottic airway (e.g. endotracheal tube, endobronchial tube, tracheostomy tube), or a supraglottic airway device (e.g. oropharyngeal airway, nasopharyngeal airway, and laryngeal mask airway).

The heating arrangement may be configured such that the inspiratory gases maintain an absolute humidity of at least 33 mg/l.

The first heating member may comprise a heating power in the range 25 to 40 Watts, optionally in the range 27 to 35 Watts, optionally in the range 29 to 33 Watts, for example approximately 31 Watts.

The first heating member may extend around the inner conduit wall.

This has been found to improve the uniformity of heating of the gases flowing within the coaxial conduit assembly.

The first heating member may spiral around the inner conduit wall.

Arranging the first heating element as a spiral around the wall of the inner conduit has been found to provide more uniform heating of the inspiratory gases flowing within the inner conduit.

The inner conduit wall may comprise a reinforcing rib extending therearound, and wherein the first heating member is embedded within the reinforcing rib.

Arranging the first heating element within the reinforcing rib has been found to improve the packing of the breathing assembly.

The first heating member may comprise two spaced apart heating elements, e.g. wires, embedded within the reinforcing rib.

This has been found improve the uniformity of heating of the gases flowing within the coaxial conduit assembly

The provision of two heating elements enables the feed and return current path to be contained within the same assembly.

The heating arrangement may comprise a second heating member configured and arranged to heat gases flowing along the second flow passage, wherein the second heating member is either positioned in the second flow passage or is embedded within the outer conduit wall.

This arrangement helps to further heat expiratory gases to reduce the build-up of water and water vapour in the second flow passage or to heat inspiratory gases to ensure that are provided to the patient at the correct temperature.

Provision of a dedicated heater for the second flow passage enables the gases within the inner and outer flow passages to be heated to separate temperatures.

The breathing assembly may be configured such that inspiratory gases are conveyed along the first flow passage and expiratory gases are conveyed along the second flow passage, in use.

In this arrangement, the second flow passage provides a thermal barrier between the inspiratory gas in the first flow passage and the ambient air, which facilitates maintaining the inspiratory gas at the required temperature.

The first heating member may be configured to heat the inspiratory gases to a temperature in the range 36° C. to 41° C., for example in the range 37° C. to 40° C.

The outer conduit wall may be at least partially formed from a water permeable material configured to allow water to flow therethrough and configured to restrict flow of respiratory gases therethrough.

The second heating member may be configured to heat the outer conduit wall to a predetermined temperature range or a predetermined temperature.

This enables the second heating member to heat the outer conduit wall to a temperature or temperature range so as to maximise its permeability to water.

The second heating member may comprise a heating power in the range 5 to 40 Watts.

The water permeable material may be configured to restrict the flow of liquid water therethrough.

Providing an outer conduit that is formed from a water permeable material that allows water to flow therethrough and that allows, but restricts, the passage of liquid water therethrough has been found to reduce the build-up of water and water vapour in the second flow passage.

An entirety of the outer conduit wall may be formed from the water permeable material.

This has been found to enhance the breathability of the outer conduit, thereby further reducing the build-up of water and water vapour in the second flow passage.

The outer conduit wall may be self-supporting.

This removes the need for additional structural members to be mounted to the outer conduit to provide the necessary structural stiffness/rigidity.

The outer conduit wall may be configured to support the inner conduit wall.

The coaxial conduit assembly may comprise a spacing arrangement between the inner conduit wall and the outer conduit wall.

The spacing arrangement may comprise at least one spacing assembly provided along the elongate length of the coaxial conduit assembly.

Each spacing assembly may comprise a plurality of spacing members positioned between the inner conduit wall and the outer conduit wall.

The plurality of spacing members may be equally spaced around the inner conduit wall.

The spacing members may project inwardly (i.e. radially inwardly) from the outer conduit wall.

The outer conduit may comprise a reinforcing arrangement.

This provides the outer conduit with improved structural stiffness/rigidity.

The reinforcing arrangement may comprise a corrugated region of the outer conduit wall.

The corrugated region may extend over an entirety of the elongate length of the outer conduit wall.

This removes the need for additional structural members to be mounted to the outer conduit to provide the necessary structural stiffness/rigidity.

The outer conduit may define an outer conduit wall having a thickness in the range 0.2 mm to 1.0 mm, optionally in the range 0.3 mm to 0.9 mm.

Providing an outer conduit wall in this range has been found to provide sufficient strength to the outer conduit whilst providing a suitably water permeable outer conduit wall.

The inner conduit wall may be insufficiently strong to be self-supporting.

The inner conduit wall may be formed from a thermoplastic elastomer.

The inner conduit wall may be configured to prevent water and water vapour from flowing therethrough.

The inner conduit may be configured to prevent liquid water and air/gases from flowing therethrough.

This arrangement enables the humidity of the inspiratory gases to be actively, rather than passively, controlled by the breathing assembly.

The inner conduit wall may comprise a reinforcing arrangement.

Incorporating a reinforcing arrangement enables the thickness of the wall of the inner conduit to be reduced, thus reducing the amount of material used in the inner conduit.

The reinforcing arrangement may comprise a reinforcing rib extending around, e.g. helically around, the inner conduit wall.

This has been found to further improve reinforcement of the inner conduit.

The breathing assembly may comprise a flow valve at or near the interface between the coaxial conduit assembly and the patient end connector, the flow valve configured to allow flow of inspiratory gases from the first flow to passage into the patient end connector and to direct expiratory gases flowing from the patient end connector to the coaxial conduit assembly into the second flow passage.

The breathing assembly may comprise a catheter mount connected to the patient end connector.

The breathing assembly may comprise a second temperature sensor at or near the patient end connector.

The second temperature sensor may be integrally formed with the patient end connector. Alternately, the second temperature sensor may be separate from, and connectable to, the patient end connector (i.e. the second temperature sensor may be reusable).

This has been found to improve the packing of the breathing assembly.

The inner conduit wall may comprise a reinforcing rib extending therearound, and wherein one or more electrical wires connected to the second temperature sensor are embedded within the reinforcing rib.

Arranging the electrical wires within the reinforcing rib has been found to improve the packing of the breathing assembly.

The breathing assembly may comprise a first temperature sensor at or near the ventilator end connector.

The first temperature sensor may be integrally formed with the ventilator end connector. Alternately, the first temperature sensor may be separate from, and connectable to, the ventilator end connector (i.e. the first temperature sensor may be reusable).

This has been found to improve the packing of the breathing assembly.

The heating arrangement may comprise a second heating member configured to heat gases flowing along the second flow passage, wherein the second heating element is either positioned in the second flow passage or is embedded within a wall of the outer conduit.

The breathing assembly may comprise an expiratory port for conveying expiratory gasses out of the breathing assembly, wherein the expiratory port is provided at or proximate to the patient end connector.

### BRIEF DESCRIPTION OF THE DRAWINGS

Embodiments will now be described with reference to the accompanying drawings, in which:

FIG. 1 is an isometric view of a part of a breathing assembly according to an embodiment;

FIG. 2 is a cross-sectional side view of the breathing assembly of FIG. 1;

FIG. 3 is a cross-sectional partial view of the breathing assembly of FIG. 1;

FIG. 4 is a cross-sectional partial view of the inner and outer conduits of the breathing assembly of FIG. 1;

FIG. 5 is a partial cross-sectional partial view of the outer conduit of the breathing assembly of FIG. 1;

FIGS. 6A and 6B are a side view and a cross-sectional side view of an inner conduit of the breathing assembly of FIG. 1.

### DETAILED DESCRIPTION OF EMBODIMENT(S)

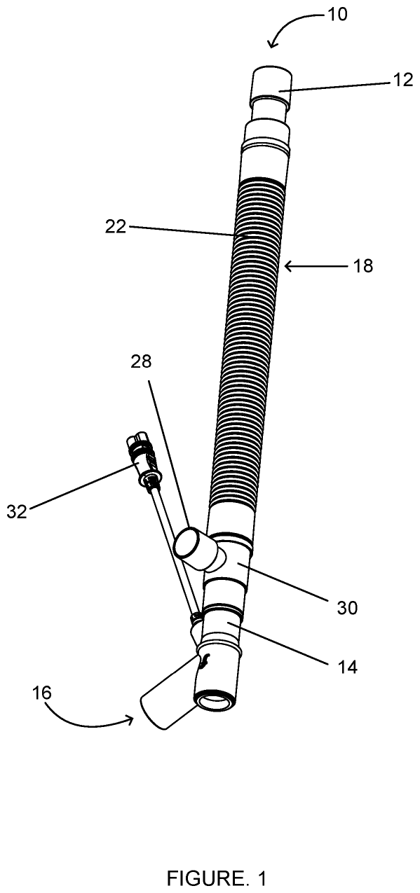

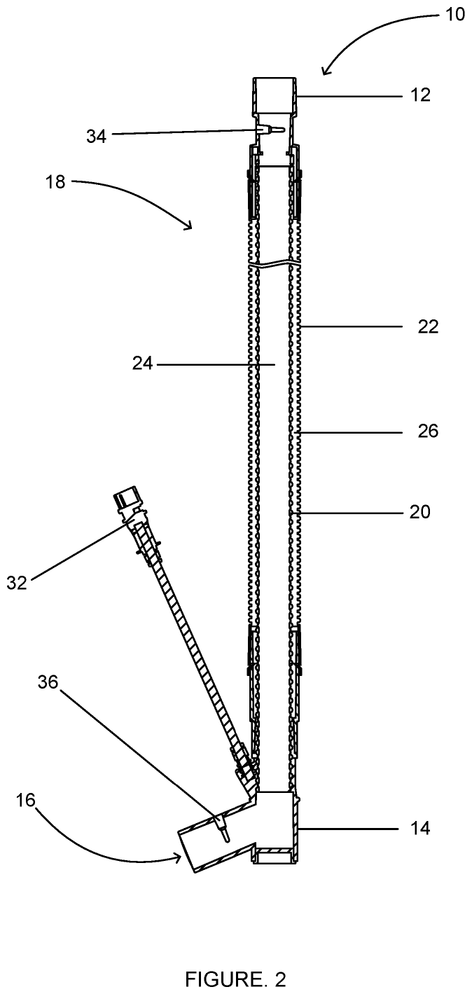

Referring firstly to FIGS. 1 and 2, a breathing assembly for supplying gases to a patient (not shown) is illustrated and is indicated generally at 10.

The breathing assembly 10 includes a patient end connector 12. Although not illustrated, the breathing assembly 10 may include a catheter mount connected to the patient end connector 12. The patient end connector 12 is configured to be connectable to an air way device. The patient end connector 12 may be configured to be connectable to a catheter mount. The catheter mount, when fitted, transports both inspiratory gases to an airway device and expiratory gases from an airway device. The patient end connector 12 may be configured to be connectable directly to airway devices such an infraglottic airway device (e.g. endotracheal tube, endobronchial tube, tracheostomy tube) or a supraglottic airway device (e.g. oropharyngeal airway, nasopharyngeal airway, and laryngeal mask airway), or any other suitable airway device.

The breathing assembly 10 includes a chamber end connector 14. Although not illustrated, the breathing assembly 10 may be connected to a humidification chamber connected to the chamber end connector 14. Put another way, the chamber end connector 14 is configured to be connectable to a humidification chamber. The chamber end connector 14 includes an inspiratory inlet 16 for receiving inspiratory gases from the humidification chamber.

The breathing assembly 10 includes a coaxial conduit assembly 18. The coaxial conduit assembly 18 extends between the patient end connector 12 and the chamber end connector 14. The coaxial conduit assembly 18 includes an inner conduit 20 and an outer conduit 22. The inner conduit 20 is defined by an inner conduit wall. The outer conduit 22 is defined by an outer conduit wall. The inner conduit 20 is arranged within the outer conduit 22 so as to define a first flow passage 24 along the coaxial conduit assembly 18 within the inner conduit 20 and a second flow passage 26 along the coaxial conduit assembly 18 between the inner conduit 20 and outer conduit 22.

The breathing assembly 10 includes an expiratory port 28. The expiratory port 28 is provided for conveying expiratory gasses out of the breathing assembly 10. In the illustrated arrangement, the expiratory port 28 is provided on a collar 30 interposed between the chamber end connector 14 and the coaxial conduit assembly 18. In alternative arrangements, the expiratory port 28 may be provided on the chamber end connector 14 or may be provided on a region of the coaxial conduit assembly 18 adjacent to, i.e. close to, the chamber end connector 14. Put another way, the expiratory port 28 may be at or proximate to the chamber end connector 14.

The breathing assembly 10 includes an electrical connector 32 for connecting to an electrical source of power. The electrical connector 32 connects the breathing assembly 10 to a control system (not shown). As will be discussed in more detail below, the electrical connector 32 is connected to various components of the breathing assembly 10 such that the components of the breathing assembly 10 can be connected to the control system and/or to provide electrical power to the components of the breathing assembly 10.

Although not illustrated, the breathing assembly 10 may include a flow valve. The flow valve is provided to allow flow of inspiratory gases from the coaxial conduit assembly 18 into the patient end connector 12 and to direct expiratory gases flowing from the patient end connector 12 to the coaxial conduit assembly 18 into the desired first or second flow passage 24, 26. Typically this flow valve is provided at the ventilator (not shown) so as to reduce complexity of the breathing assembly. Alternatively, the flow valve may be provided at or near the interface between the coaxial conduit assembly 18 and the patient end connector 12.

In the illustrated arrangement, the breathing assembly 10 is configured such that inspiratory gases are conveyed along the first flow passage 24 and expiratory gases are conveyed along the second flow passage 26, in use. In this arrangement, the second flow passage 26 (i.e. the outer flow passage) provides a thermal barrier between the inspiratory gas in the first flow passage 24 and the ambient air, which facilitates maintaining the inspiratory gas at the required temperature (e.g. around 37° C.). It will be appreciated, however, that in alternative arrangements, the breathing assembly 10 may be configured such that inspiratory gases are conveyed along the second flow passage 26 and expiratory gases are conveyed along the first flow passage 24, in use.

The breathing assembly 10 includes a first temperature sensor 36. The first temperature sensor 36 is a temperature sensor. The first temperature sensor 36 is positioned at or near the chamber end connector 14. In the illustrated embodiment, the first temperature sensor 36 is integrally formed with the chamber end connector 14. In alternative arrangements, however, it will be appreciated that the first temperature sensor 36 may be removable, e.g. disposable. The first temperature sensor 36 is connected to the electrical connector 32. Put another way, the first temperature sensor 36 is connectable to a control system (not shown).

The breathing assembly 10 includes a second temperature sensor 34. The second temperature sensor 34 is a temperature sensor. The second temperature sensor 34 is positioned at or near the patient end connector 12. In the illustrated embodiment, the second temperature sensor 34 is integrally formed with the patient end connector 12. In alternative arrangements, however, it will be appreciated that the second temperature sensor 34 may be removable, e.g. disposable. The second temperature sensor 34 is connected to the electrical connector 32. Put another way, the second temperature sensor 34 is connectable to a control system (not shown) and/or an electrical source of power via the electrical connector 32. The second temperature sensor 34 is connected to the electrical connector 32 via one or more wires (not shown) extending along the coaxial conduit assembly 18, for example embedded within a wall of the inner conduit 20 or outer conduit 22.

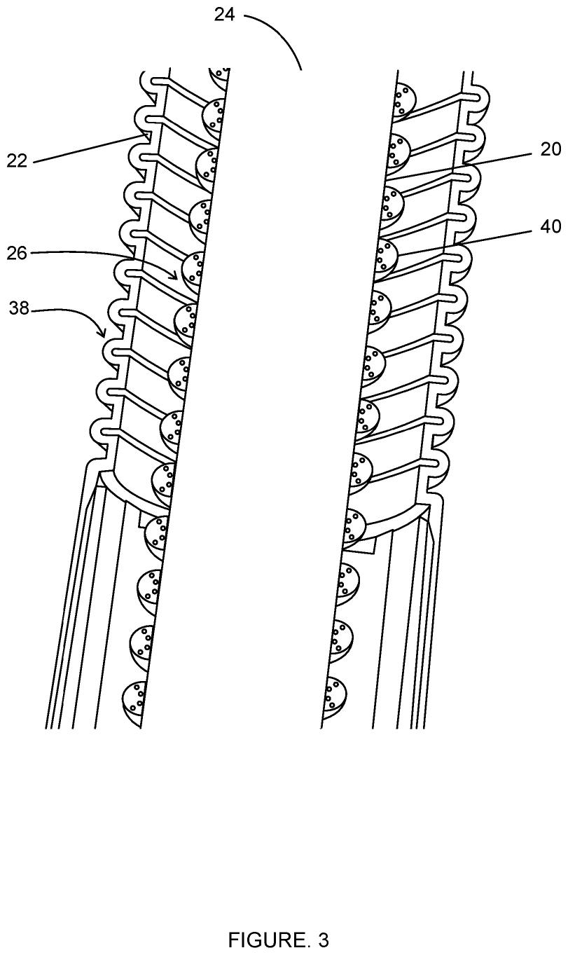

Referring now to FIG. 3, the coaxial conduit assembly 18 includes inner and outer conduits 20, 22. The outer conduit wall 22 is at least partially formed from a water permeable material. A water permeable material is one that is configured to allow water to flow therethrough. It will be appreciated that a majority of the outer conduit wall 22 may be formed from the water permeable material. In the present arrangement, substantially all, e.g. an entirety, of the outer conduit wall 22 is formed from the water permeable material.

The water permeable material may also be configured to restrict flow of liquid water therethrough and/or is configured to restrict flow of gases therethrough. Forming the outer conduit wall from a material allowing water to flow therethrough and that allows, but restricts, the passage of liquid water and/or gases therethrough has been found to reduce the build-up of water and water vapour in the second flow passage. The restriction of the passage of liquid water and/or gases therethrough are lowered to within the clinical requirements of ventilator machine requirements, as defined in the standard ISO5367 for Anaesthetic and respiratory equipment—Breathing sets and connectors.

The outer conduit wall 22 is self-supporting. Put another way, the outer conduit 22 is sufficiently strong, or sufficiently study, so as to be self-supporting. In this way, no separate reinforcing members are required to be mounted/attached to the outer conduit 22 (which could be detrimental to the breathability of the outer conduit 22). This helps to increase the area of the outer conduit 22 that is water permeable. The outer conduit 22 defines an outer conduit wall having a thickness in the range 0.2 mm to 1.0 mm, optionally in the range 0.3 mm to 0.9 mm.

The outer conduit wall 22 includes a reinforcing arrangement. Put another way, the outer conduit wall 22 is arranged so as to define a reinforcing formation. This provides the outer conduit 22 with improved structural strength. The reinforcing arrangement is provided in the form of a corrugated region 38 of the outer conduit (i.e. the conduit wall of the outer conduit is corrugated). In the illustrated arrangement, the corrugated region extends over an entirety of the elongate length of the outer conduit 22. The corrugations in the outer conduit 22 work to increase the overall surface area of the water permeable outer conduit 22, thus increasing the area through which water within outer flow path can pass out of the breathing assembly 10.

The water permeable material of the outer conduit 22 is formed from an amphiphilic material. The water permeable material is formed from an amphiphilic block co-polymer. Put another way, the water permeable material is formed from a hydrophobic and hydrophilic block co-polymer. An example of one such material is a hydrophobic and hydrophilic poly (ethylene oxide) based block co-polymer. Further examples is suitable water permeable materials are: Nafion®; Sympatex®; Arnitel®; Diaplex®; and water permeable Hytrel®, and it will be appreciated that any suitable water permeable material may be used.

In some arrangements, the water permeable material may be configured to absorb water vapour and liquid water. This has been found to further enhance the permeability of the outer conduit 22 to water vapour and liquid water, thereby further reducing the build-up of water and water vapour in the second flow passage.

In the present arrangement, the inner conduit wall 20 is insufficiently strong to be self-supporting. Put another way, the conduit wall of the inner conduit may have a low wall thickness (i.e. less than the outer conduit 22 wall thickness) such that the inner conduit 20 is insufficiently strong, or insufficiently study, so as to be self-supporting.

The inner conduit wall 20 is configured to prevent water and water vapour from flowing therethrough. The inner conduit wall 20 may be configured to prevent gases from flowing therethrough. As there is no transmission of moisture between the inspiratory and expiratory gases, the humidity of the inspiratory gases is able to be actively, rather than passively, controlled by the breathing assembly 10. In the present arrangement, the inner conduit wall 20 is formed from a thermoplastic elastomer. However, in alternative arrangements, it will be appreciated that any suitable material may be used such as thermoplastic extrudable polymers, for example polyethylene or polyurethane.

The inner conduit wall 20 is provided with a reinforcing arrangement. Incorporating a reinforcing arrangement on the inner conduit 20 enables the thickness of the wall of the inner conduit 20 to be reduced, thus reducing the amount of material used in the inner conduit 20. The reinforcing arrangement is provided in the form of a reinforcing rib 40. The reinforcing rib 40 extends around the inner conduit 20. In the illustrated arrangement, the reinforcing rib 40 extended helically (i.e. spirals) around the inner conduit 20.

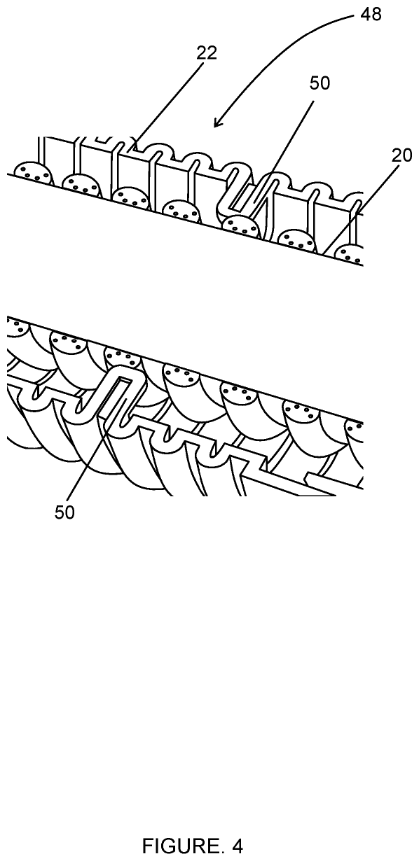

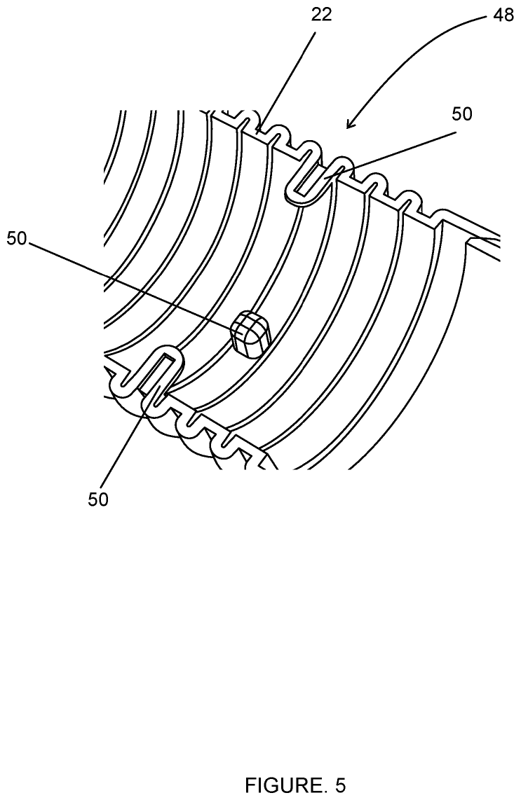

Referring now to FIGS. 4 and 5, the coaxial conduit assembly 18 includes a spacing arrangement between the inner conduit wall 20 and the outer conduit wall 22. The spacing arrangement includes a spacing assembly 48. The spacing arrangement may be provided with an array of spacing assemblies 48 along the elongate length of the coaxial conduit assembly 18.

Each spacing assembly 48 has a plurality of spacing members 50 positioned between the inner conduit 20 and the outer conduit 22. The plurality of spacing members 50 are equally spaced around the inner conduit 20. In the illustrated arrangement, four spacing members 50 are provided, but it will be appreciated that any suitable number of spacing members 50 may be provided such as three, five or more spacing members 50.

The spacing members 50 are configured and arranged to support the inner conduit 22. Put another way, the outer conduit 22 is configured to support the inner conduit 20. In the illustrated arrangement, the spacing members 50 project inwardly (i.e. radially inwardly) from the outer conduit wall 22. In alternative arrangements, the spacing members 50 may be separate from, but attached to, the inner conduit 20 and/or outer conduit 22.

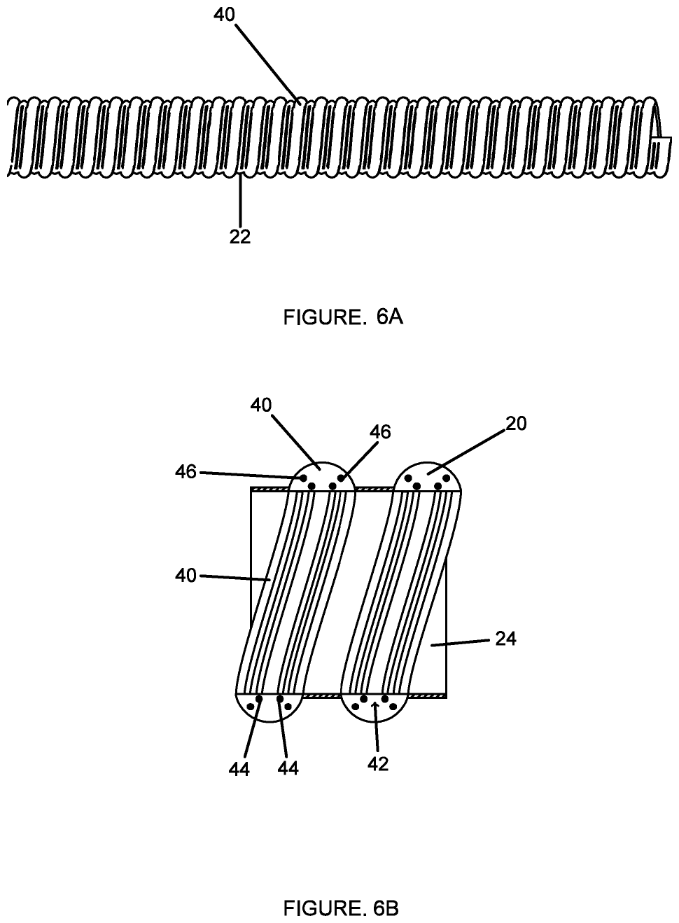

Referring now to FIGS. 6A and 6B, the breathing assembly 10 includes a heating arrangement for heating gases flowing along the coaxial conduit assembly 18.

The heating arrangement includes a first heating member 42. The first heating member 42 is configured and arranged to heat gases flowing along the first flow passage 24 and the second flow passage 26. The first heating member is configured to heat the inspiratory gases to a predetermined temperature of temperature range. The heating arrangement is configured such that the inspiratory gases maintain an absolute humidity of at least 33 mg/l. In the present arrangement, the first heating member is configured to heat the inspiratory gases to a temperature in the range 36° C. to 41° C., for example in the range 37° C. to 40° C. In the arrangement shown, the first heating member is configured to heat the inspiratory gases in the first flow passage to a temperature in the range 36° C. to 41° C., for example in the range 37° C. to 40° C. The first heating member comprises a heating power in the range 25 to 40 Watts, or in the range 27 to 35 Watts, optionally in the range 29 to 33 Watts, for example approximately 31 Watts.

The first heating member 42 is provided in the form of two spaced apart heating elements 44, e.g. wires. The first heating member 42 is embedded with the wall of the inner conduit wall 20. The first heating member 42 extends around, e.g. spirals around, the inner conduit wall of the inner conduit 20. In the illustrated arrangement, the first heating member 42 is embedded within the reinforcing rib 40. Put another way, the inner conduit 20 includes a reinforcing rib 40 having the first heating member 42 therein.

One or more electrical wires 46 are embedded in the wall of the inner conduit wall 20. The electrical wires 46 provide an electrical connection between the second temperature sensor 34 and the electrical connector 32. In the illustrated embodiment, two electrical wires 46 are embedded in the wall of the inner conduit 20. The electrical wire(s) 46 extend around, e.g. spiral around, the inner conduit wall of the inner conduit 20. In the illustrated arrangement, the one or more electrical wires 46 are embedded within the reinforcing rib 40. It will be appreciated in alternative arrangements that the one or more electrical wires 46 may be embedded in the wall of the inner conduit 20 but not within the reinforcing rib 40 of the inner conduit 20, or may be positioned within the second flow passage 26.

The heating arrangement may include a second heating member. The second heating member may be configured to heat gases flowing along the second flow passage 26. Provision of a dedicated heater for the second flow passage 26 enables the gases within the inner and outer flow passages 24, 26 to be heated to separate temperatures. The second heating member is configured to heat the outer conduit wall to a predetermined temperature range or a predetermined temperature. The water permeability of the material forming the outer conduit 22 will be dependent upon the temperature of said material. The provision of a heater configured to heat the wall of the outer conduit 22 enables the second heating member to heat the wall of the outer conduit 22 to a temperature or temperature range so as to maximise its permeability to water. It will be appreciated that the second heating element may either be positioned in the second flow passage 26 or may be embedded within a wall of the outer conduit 22. The second heating member comprises a heating power in the range 5 to 35 Watts.

Although not illustrated, it will be appreciated that the breathing assembly may be connected to a ventilator and a humidification chamber as a part of a breathing system. In such breathing systems, the humidification chamber will be arranged to be between the ventilator and the chamber end connector 14.

During inspiration, the ventilator (not shown) is vented through a limb/conduit to the humidification chamber. The inspiratory gases flowing through the humidification chamber are heated and humidified. The inspiratory gases then flow through the chamber end connector 14 and into the coaxial conduit assembly 18. The inspiratory gases are directed to flow along the inspiratory flow path (e.g. along the first flow passage 24 in the illustrated embodiment) and to the patient end connector 12. The inspiratory gases flow through the patient end connector 12 and are conveyed to a patient, e.g. via a catheter mount.

During expiration, expiratory gases are conveyed from the patient, e.g. via a catheter mount, to the patient end connector 12. The expiratory gases are then directed to flow along the expiratory flow path (e.g. along the second flow passage 26 in the illustrated embodiment) by the ventilator valve sequence inhibiting flow in the inspiratory path, but also optionally by a flow valve in the patient end connector. Expiratory gases then flow along the coaxial conduit assembly 18 and flow out of the breathing assembly 10 through the expiratory port 28, releasing the expiratory gasses to the atmosphere.

Although the teachings have been described above with reference to one or more preferred embodiments, it will be appreciated that various changes or modifications may be made without departing from the scope as defined in the appended claims.

1. A breathing assembly for supplying gases to a patient, the breathing assembly comprising:
a patient end connector configured to be connectable to an airway device;
a chamber end connector configured to be connectable to a humidification chamber and comprising an inspiratory inlet for receiving humidified inspiratory gases from the humidification chamber; and
a coaxial conduit assembly extending between the patient end connector and the ventilator end connector and comprising inner and outer conduits defined by inner and outer conduit walls, respectively, the inner conduit wall arranged within the outer conduit wall so as to define a first flow passage along the coaxial conduit assembly within the inner conduit and a second flow passage along the coaxial conduit assembly between the inner conduit and outer conduit,
wherein the breathing assembly is configured such that inspiratory gases are conveyed along the first flow passage and expiratory gases are conveyed along the second flow passage, in use, and
wherein the outer conduit wall is at least partially formed from a water permeable material configured to allow water to flow therethrough.

2. The breathing assembly according to claim 1, wherein the water permeable material is configured to allow flow of liquid water therethrough and configured to restrict flow of respiratory gases therethrough.

3. The breathing assembly according to claim 1, wherein substantially all of the outer conduit wall is formed from the water permeable material.

4. (canceled)

5. The breathing assembly according to claim 1, wherein the outer conduit wall comprises a corrugated region.

6. (canceled)

7. (canceled)

8. The breathing assembly according to claim 1, wherein the coaxial conduit assembly comprises a spacing arrangement between the inner conduit wall and the outer conduit wall, and wherein the spacing arrangement comprises at least one spacing assembly provided along the elongate length of the coaxial conduit assembly, and wherein the or each spacing assembly comprises a plurality of spacing members positioned between the inner conduit and the outer conduit.

9. The breathing assembly according to claim 1, wherein the water permeable material is formed from an amphiphilic material.

10. The breathing assembly according to claim 9, wherein the water permeable material is formed from a hydrophobic and hydrophilic block co-polymer.

11. (canceled)

12. (canceled)

13. The breathing assembly according to claim 1, wherein the inner conduit comprises a reinforcing arrangement comprising a reinforcing rib extending around the inner conduit wall.

14. The breathing assembly according to claim 1, comprising a heating arrangement configured and arranged to heat inspiratory gases flowing along the first flow passage, wherein the heating arrangement comprises a first heating member embedded within the inner conduit wall.

15. The breathing assembly according to claim 14, wherein the first heating member extends around the inner conduit wall.

16. The breathing assembly according to claim 14, wherein the first heating member is configured and arranged to heat inspiratory gases flowing along the first flow passage and to heat expiratory gases flowing along the second flow passage.

17. (canceled)

18. The breathing assembly according to claim 1, wherein the heating arrangement comprises a second heating member configured to heat gases flowing along the second flow passage, wherein the second heating element is either positioned in the second flow passage or is embedded within the outer conduit wall.

19. (canceled)

20. A breathing assembly for supplying gases to a patient, the breathing assembly comprising:
a patient end connector configured to be connectable to an air way device;
a ventilator end connector configured to be connectable to a humidification chamber and comprising an inspiratory inlet for receiving humidified inspiratory gases from the humidification chamber;
a coaxial conduit assembly extending between the patient end connector and the ventilator end connector and comprising inner and outer conduits defined by inner and outer conduit walls, respectively, the inner conduit wall arranged within the outer conduit wall so as to define a first flow passage along the coaxial conduit assembly within the inner conduit wall and a second flow passage along the coaxial conduit assembly between the inner conduit wall and outer conduit wall, wherein the first flow passage and second flow passage each convey one of inspiratory gases or expiratory gases, in use; and a heating arrangement for heating gases flowing along the coaxial conduit assembly,
wherein the heating arrangement comprises a first heating member embedded within an inner conduit wall, and wherein the first heating member is configured and arranged to heat gases flowing along the first flow passage and the second flow passage.

21. The breathing assembly according to claim 20, wherein the first heating member extends around the inner conduit wall.

22. The breathing assembly according to claim 20 wherein the inner conduit wall comprises a reinforcing rib extending therearound, and wherein the first heating member is embedded within the reinforcing rib.

23. (canceled)

24. The breathing assembly according to claim 20, wherein the breathing assembly is configured such that inspiratory gases are conveyed along the first flow passage and expiratory gases are conveyed along the second flow passage, in use.

25. The breathing assembly according to claim 24, wherein the first heating member is configured to heat the inspiratory gases to a temperature in the range 36° C. to 41° C.

26. The breathing assembly according to claim 20, wherein the heating arrangement comprises a second heating member configured and arranged to heat gases flowing along the second flow passage, and wherein the second heating member is either positioned in the second flow passage or is embedded within the outer conduit wall.

27. The breathing assembly according to claim 20, wherein the outer conduit wall is at least partially formed from a water permeable material configured to allow water to flow therethrough and configured to restrict flow of respiratory gases therethrough.

28. (canceled)

29. (canceled)

30. The breathing assembly according to claim 20, comprising a first temperature sensor at or near the ventilator end connector and/or comprising a second temperature sensor at or near the patient end connector.
